(19) United States
(12) Patent Application Publication
(10) Pub. No.: US 2012/0277594 A1
(43) Pub. Date: Nov. 1, 2012

(54) MENTAL HEALTH AND WELL-BEING

(76) Inventor: Timothy R. Pryor, Sylvania, OH (US)

(21) Appl. No.: 13/544,180

(22) Filed: Jul. 9, 2012

Related U.S. Application Data
(63) Continuation-in-part of application No. 12/358,404, filed on Jan. 23, 2009.
Application No. 12/358,404 is a continuation-in-part of application No. 12/754,285, filed on Apr. 5, 2010.
(60) Provisional application No. 61/506,212, filed on Jul. 11, 2011.

(51) Int. Cl. A61B 5/11; A61B 6/00; A61B 5/12; H04N 7/18; A61B 3/02

(52) U.S. Cl. 600/476; 348/143; 600/595; 600/558; 600/559; 348/E07.085

(57) ABSTRACT

Mental illness is a huge burden on those afflicted with it, as well as their caregivers and represents a major cost to society as a whole. The invention herein is concerned with methods and apparatus to improve the well-being of persons afflicted with mental illness, as well as to provide early and better diagnosis. It is felt that in some cases such diagnosis can lead to a potential for eliminating the worst effects of the illness altogether, by allowing intervention before a first psychotic episode occurs. The invention uses sensors, primarily 2D or 3D cameras and voice recognition to observe persons in many instances during ordinary activity such as playing video games, chatting on the internet or other such computer based activity.

### CROSS REFERENCE TO RELATED APPLICATIONS

This application is a continuation-in-part of prior application Ser. No. 12/358,404, filed Jan. 3, 2009, and entitled “Motivation and Enhancement of Physical and Mental Exercise, Rehabilitation and Health.” This application is also a continuation-in-part of prior application Ser. No. 12/754,285, filed Apr. 5, 2010, and entitled “Camera Based Interactive Exercise.” This application also claims the benefit of U.S. Provisional Application No. 61/506,212, filed Jul. 11, 2011, and entitled “Mental Health and Well Being.” The above disclosures are hereby incorporated by reference in their entirety.

Also incorporated by reference are the disclosures of (a) U.S. Pat. No. 8,189,053, entitled “Camera Based Interaction and Instruction” and (b) U.S. Pat. No. 8,194,924, entitled, “Camera Based Sensing In Handheld, Mobile, Gaming Or Other Devices.”

### FIELD OF THE INVENTION

The invention herein is concerned with methods and apparatus to improve the well-being of persons afflicted with mental illness, as well as to provide earlier and/or better diagnosis of the illness and improved intervention and reduction of its effects. It is felt that in some cases such early diagnosis can lead to a potential for eliminating the worst effects altogether, by allowing intervention before a first psychotic episode occurs and perhaps forestalling it entirely or at least mitigating the effects thereof. The invention uses sensors, primarily 2D or 3D cameras and voice recognition to observe persons in many instances during ordinary activity such as playing video games, chatting on the internet or other such computer based activity.

### BACKGROUND OF THE INVENTION

Mental illness is a huge burden on those afflicted with it, as well as their caregivers and represents a major cost to society as a whole.

In the last 15 years or so, new antipsychotic drugs and better methods of care in the community have made it possible for many afflicted persons to remain at home or in residential housing rather than hospitals. However, the choice of medication is often based on limited information or general lack of knowledge both in the medical community or of the person in question. This can and does often result in inadequate care and can lead to intolerable side effects, (weight gain, diabetes, drugged feelings, etc.) which in turn can dissuade the patient from taking the drugs at all or to skip doses. This often has unfortunate consequences, and motivation to take the drugs is a key issue in the community, which in turn depends on the efficacy of the medication to make the whole mental health system function.

In addition, it is often difficult to properly provide care in the community, and in many locales care is sporadic, or even near non-existent. Huge burdens fall upon the caregiver, which is often aging parents, understaffed group homes, or in some cases no one at all.

### SUMMARY OF THE INVENTION

In the above mentioned co-pending applications and others related thereto, I have discussed movement and location sensing and voice recognition for use in diagnosis exercise, and social interaction as well as a general aid to well-being of persons with mental illnesses.

In this application I wish to expand on the theme of previous applications and disclose further apparatus and methods by which well-being of mentally ill patients can be improved, and perhaps costs for treating the illness, reduced.

A preferred embodiment of the invention contains features described in the previous applications such as electro-optical detection of position or motion of points on persons or objects, facial expressions, gestural expressions, and voice recognition as well as speech output and a large video display.

A suitable commercial device today to serve as a basis for the invention is Microsoft® Kinect™ running on the Microsoft® Xbox™ or a PC with a HDTV display and audio speakers. This system or a portable or handheld variant thereof disclosed herein (for example running on a smartphone) is used to improve the well-being of a person already afflicted with mental illness in a variety of ways. Alternatively (or in addition), it may be used to monitor a person suspected of having mental illness in order to determine if additional tests or intervention is warranted. Such monitoring could even be in the form of a screening test done on the general population, for example while playing video games and interacting over the internet as many young people do. Thanks to such early screening made possible by the invention, intervention may be provided (e.g., using anti-psychotic drugs) before the person has a first psychotic episode, and I believe potentially reducing greatly the chance to develop severe mental illness.

The invention may aid diagnosis not only of the presence of mental illness but of the type and characteristics thereof.

It is important to note that young people are often the ones who become afflicted by schizophrenia either late in their teens or early in their 20s and thus such screening could be highly desirable. The need for earliest and best possible identification of potential problems is essential and this is a substantial benefit the invention offers.

The invention, by performing the monitoring function disclosed herein, can also help with continued modification of treatment plans and aid compliance with same. A positive result may be experienced in cases where the use of the invention may allow dosage to be lowered for a patient, thereby saving money and generally being more acceptable for the patient who then experiences fewer deleterious side effects.

There could be benefit to other brain related disorders as well, such as Alzheimer's, though the invention in a way is better suited to youth who are accustomed to using computer systems.

The invention is not solely concerned with diagnosis or treatment. It is also an aid to the instant and continued well-being of afflicted persons. A test run in part with the inventor's support on a small sample (20) of persons with persistent mental illness resulted in data just now being analyzed which indicates that the activity of video games useful in such systems can greatly improve well-being and health of this population. Implicit in this is the possibility of new methods for care in the community.

It is a goal of the invention to improve care of mentally ill persons via feedback during computer based activity.

It is also a goal to provide methods and apparatus for diagnosis of mental illness.

It is another goal to provide methods and apparatus for use in screening for mental illness and symptoms thereof.

It is a further goal of the invention to provide a method by which some persons can receive lower doses of medication having deleterious side effects.

It is a goal of the invention to improve social skills of mentally ill persons.

It is also a goal of the invention to provide methods for possible mitigation or even elimination of mental illness in certain individuals.

It is a goal of the invention to increase the amount of reliable data on which a diagnosis of a potential illness can be made, and to allow this to be done at an early age.

It is also a goal of the invention to provide means for monitoring the state of medication of persons.

It is a further goal of the invention to provide a method for rapid intervention to aid mentally ill persons.

It is another goal to automatically provide, via the computer of the invention, therapeutic signals suggestions and the like for the person using the device.

It is a further goal of the invention to provide a sensing device for position and movement which can achieve accurate results aiding the use of clouds of data points.

Further features and advantages of the present invention will be set forth in, or apparent from, the detailed description of preferred embodiments thereof which follows.

### BRIEF DESCRIPTION OF THE DRAWINGS

FIG. 1 illustrates a preferred embodiment for well-being enhancement in game or social interaction.

FIG. 2 illustrates one example of a diagnostic process.

FIG. 3 illustrates an iPad2 or other tablet based embodiment used as a personal care assistant including detection of finger and hand position or movement.

FIG. 4 illustrates use of a cell phone for care and diagnosis.

FIG. 5 illustrates care methods employing computer processing and electro-optical sensing devices.

FIG. 6 illustrates improvement of well-being of person in one example.

### EMBODIMENTS OF THE INVENTION

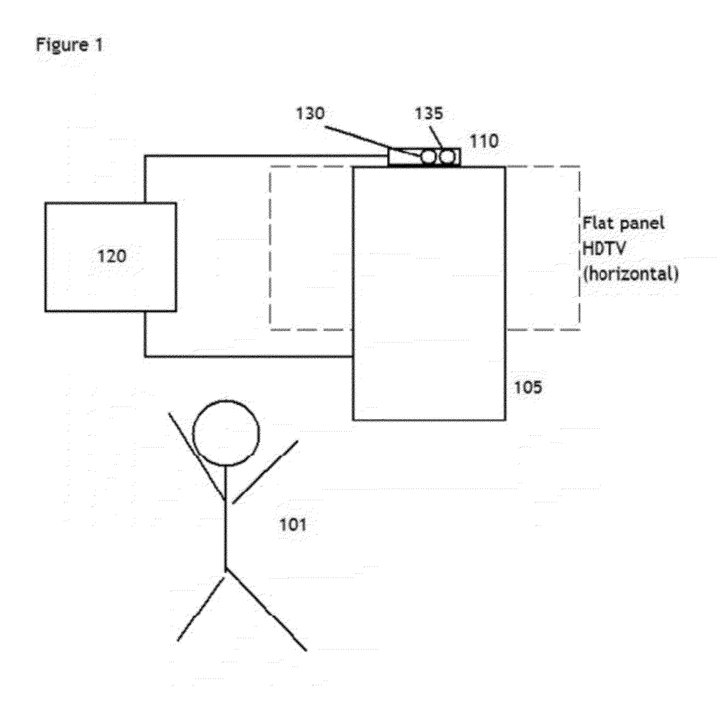

FIG. 1 illustrates one embodiment of the invention, which contains features described in my previous applications referenced above such as detection of position or motion of points on persons or objects, facial expressions or other gestural expressions, and voice recognition as well as speech output and a large video display, including a vertically oriented display.

One commercial device today suitable for use with the invention is the Microsoft® Kinect™ unit 110 controlled by the Microsoft® Xbox™ or a PC such as 120. Such devices may shrink in size in the future and be enabled on portable devices such as tablets or smartphones as well.

Typical is a HDTV Display dotted lines horizontal. Shown here however, also is disclosed in prior applications, the HDTV display 105 may advantageously be oriented long axis vertical in drawing to allow lifelike (or seemingly lifelike) presentation of a partner or coach or caseworker. Sensor processing can be in the computer unit shown or associated with the sensor proper or both.

A goal of the invention is to use this system (and/or a portable or handheld variant thereof also disclosed) to improve the well-being of a person already afflicted with mental illness or to monitor their state. The invention can also monitor a person suspected of having mental illness in order to determine if additional tests or intervention is warranted. Such monitoring could even be in the form of a screening test done on the general population, for example, while playing video games as many young people do. It is important to note that young people are often the ones who become afflicted by schizophrenia either late in their teens or early in their 20s and thus such screening could be highly desirable.

As shown in the Figure, a person 101 stands or sits in front of the TV display 105 and is observed by a sensor such as the sensor 110 having suitable camera and microphone capabilities which is able to determine (using a computer within the sensor or in a larger computer such as the computer 120 controlling the TV) their position, motion, facial expression, and to recognize things the person says. Many sensed characteristics of the person are desired, but in some activities only a subset may be used. The TV is used to both display text and graphical data to the person (including image data of themselves or caregivers or group members for example), as well as to provide audio information via loudspeakers to the person.

The sensor unit 110 has a 3D range sensing camera 130 and a conventional webcam 135. It also may have a microphone section not shown, but also able to be inputted to the computer 120, whose output can drive the TV display 105 having a display and speakers associated therewith.

The person can be asked questions by an image displayed on the screen, or the person may respond to images by performing motions such as jumping, dancing, shooting or other motions. The person on the screen may be a computer graphic able to respond instantly, or a canned video clip provided first or in response to the person's actions, or it may be a actual person such as a remotely located caregiver or medical person, assuming the computer is connected to the internet or other transmission medium.

### Diagnosis and Monitoring

In terms of watching the responses using a program in the computer or a remote computer, one would typically be looking for a response that deviated from some norm of the general population. This analysis can be done automatically or in conjunction with a doctor, nurse or social worker who can look at the results often from a remote location. Such analysis can be each time, or periodically such as daily weekly or other interval.

If someone was known a priori to have a certain sort of affliction the program or medical team could be looking for that response and to determine its degree. This could have substantial value in medication programs because often times the medication taken by mentally ill people is in essence a “cocktail”—one that is created by the individual psychiatrist to try and meet what he perceives as the needs of the person. The resulting dose may contain both medicines to reduce the psychotic effects but also other medicines to reduce the side effects of the anti-psychotic medicine. It is indeed often a cocktail and one that needs as much monitoring as possible in order to optimize. The invention herein gives the possibility of doing it automatically using computer-aided analyses of motions, audio responses, facial expressions and gestures, optionally or alternatively allowing a remote caregiver or medical person to analyze the person directly.

Where desired a video clip (generally including audio) taken of the person may be transmitted, allowing remote practitioners to directly observe the person during activity, and for many cases pre-screened activity which may be suspect. In other words the computer program analyzes the data, such that only data of interest is transmitted. Or if all the data is transmitted, a program at the receiving site can pre-screen the information to present potential problem areas to the medical team. The effect of this is to greatly increase the reach of the medical community with respect to the clients who are ill or may become ill. In addition, it is anticipated that the caregiver, which often is a parent, can input data as well either visually, vocally or typing it in to get the data to the medical support people so they can determine the effectiveness of the medicine used—or the need for medicine at all, and if so what kind.

I believe this has substantial potential value in many cases resulting in better adjustment of medication, and possibly lower doses, having fewer side effects for the people, which would then make them more willing to stay on their medication, which is often a major problem.

In the course of a normal game such as a dancing game for example, one might look for movements which would indicate a movement disorder. In a shooting game or other such action game, one might look for excessive activity (verbal or movement) such as might be experienced by a bipolar person in a manic state. For example, in the latter case if one knew that the person in his or her present state of medication was tending toward a manic state, one would know that the dose would have to be further monitored and potentially increased to prevent or mitigate this.

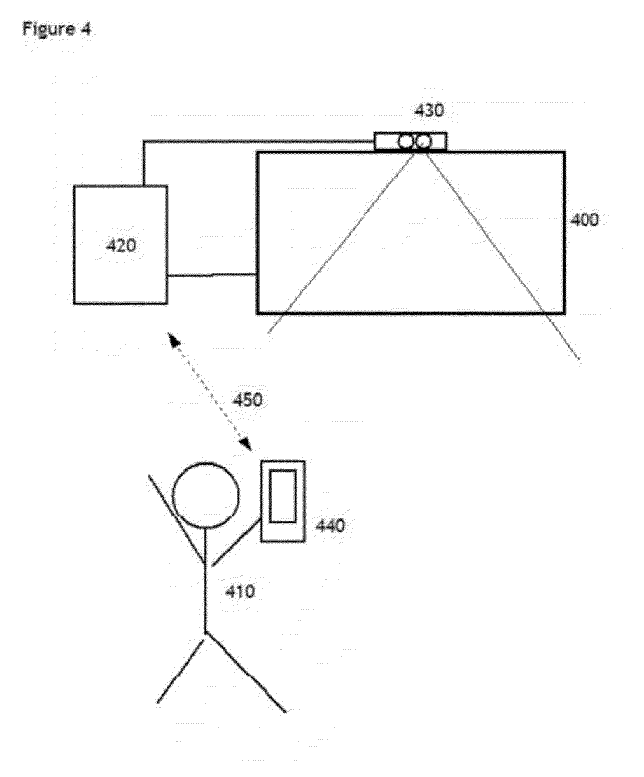

As noted above, one can screen the general population, particularly effective with young persons known to play video games. Other persons can be so screened by simply using the principles herein to monitor their activity, for example on the internet or while watching TV. In the latter case for example sensing of movement and voice like FIG. 4 might be contained within the TV remote control, or using electro-optical sensor units that use gestures which is a coming feature resembling FIG. 1.

As noted, the software incorporated into a game or activity may be used according to the invention for diagnosis. For example one can monitor any desired variables as mentioned above. I note that one also can first utilize a stimulus such as looking at lights, either computer generated from the TV or from some other lights in the room. Or one can listen to special sounds from the TV or otherwise, a social situation on the screen, or a reading of a passage. Certain types of mental illness may respond to such stimuli and become manifested in a way they can be sensed and determined to be present.

The invention comprehends that one could purposely excite the person in a manner that would cause some behavior to be observed.

It should also be noted that the stimuli could be provided by a drug, which might be provided in a trace-like amount to only act to bring on a small amount of the abnormal activity, if present. This drug could be alcohol or some other type of “normal” drug, or a drug especially created for the purpose.

The invention may aid diagnosis not only of the presence of mental illness but of the type and characteristics thereof. This may be achieved by monitoring, for example, the responses of the person: verbal, facial, gestural or other in response to stimuli or situations displayed or vocalized by the system of the invention.

### Prevention of Mental Disease or Complications thereof

There are many references dealing with what is called the first psychotic episode. Two for example are http://www.ncbi.nlm.nih.gov/pubmed/20347270 and http://www.ncbi.nlm.nih.gov/pubmed/17603411. It is of interest to consider the abstract of http://www.ncbi.nlm.nih.gov/pubmed/10598294 reproduced in the following paragraph:

“A first psychotic episode includes a wide range of disorders with different outcomes: schizophrenia, bipolar disorder, schizophreniform disorder, schizoaffective disorder, drug-induced psychosis, brief reactive psychosis, organic psychoses and delusional disorder. The course and outcome of a first psychotic episode is greatly dependent on its initial management. Major clinical, etiopathogenic and therapeutic advances have been achieved in this field and have allowed specific management strategies to be adopted. The primary task of therapists involved in the management of patients who have experienced a first episode of psychosis is promotion of recovery and prevention of secondary morbidity, relapse and persistent disability. The main guidelines of an early psychosis management are: —to keep in mind that early psychosis is not early schizophrenia. Thus, clinicians and therapists should avoid an early diagnosis of schizophrenia. Diagnosis in early psychosis can be highly unstable. A diagnosis of schizophrenia, with its implications of pessimism, relapse and disability, does not contribute anything positive in terms of guiding treatment. On the contrary, such a diagnosis may damage the patient and family by stigmatizing them and affecting the way they are viewed and managed by healthcare professionals.—To integrate biological, psychological and social interventions: effective medications is useful in reducing the risk of relapse, but is not a guarantee against it. Psychological and social interventions can greatly help promote recovery.—To tailor the various strategies to meet the needs of an individual: as an example, it is important to formulate appropriate strategies for the different stages of the illness (prodromal phase, acute phase, early recovery phase and late recovery phase) because patients have different therapeutic needs at each stage.—In the acute treatment, not to concentrate on short-term goals in indicating antipsychotic treatment: prescribing principles for first-episode psychosis are to maximize benefit and minimize side effects because the first experience of medication may influence a patient's future attitudes of therapy of all types. Effective strategies which may reduce long-term morbidity and improve recovery are currently available but their implementation is too often delayed. The time lag between the onset of symptoms and the start of treatment can be many months or years and this delay can have serious consequences. The critical period of the first 2-5 years after the first psychotic episode is a time of maximum vulnerability and of maximum opportunity. Consequently, actions should be undertaken to promote early recognition and assistance in psychotic disorders: understanding of the factors that may cause delay in treatment can help minimize this problem and lead to the initiation of appropriate treatment at the earliest opportunity. Training the general practitioners who have an important part to play in the early recognition is also of crucial importance.”

Clearly the more information that can be available at an early date, the better for the patient. Furthermore it is a hypothesis of the inventor that if one can detect abnormalities early, before a person experiences a first psychotic episode, one may provide intervention which could prevent it from occurring and thus the debilitating effects of mental illness itself. I believe the invention may provide the means to do this, as disclosed herein. At a minimum it should aid the design of therapy for individuals, and should further aid in screening for those who are at risk.

For example the invention comprehends monitoring gestures, movements, and voice and expressions. A central premise with one aspect of the invention is that the problems can be identified much more rapidly, providing benefits as implied in the article above. In this manner we may determine, at a pre-first episode time, the subject's possibility for schizophrenia, bi-polar or some other mental illness. Such determination can be based on many factors, some examples of which are discussed below.

If it is felt that a psychotic episode could happen, and particularly if there is a family history involved, one might begin therapy and increase the frequency of monitoring using the invention. If one or both indicate continuing risk, the person may be provided a low dose of an antipsychotic drug and monitoring via the invention and therapy continued. If the level of the subject's potential psychotic activity is rising, additional drugs can be given and in this manner a first episode psychosis may (at least in some cases I feel) be prevented from occurring at all, thereby decreasing brain damage and mitigating effects of the illness.

It should be noted that the invention comprehends that one could develop new drugs to aid in stimulating or otherwise helping the person in order to achieve an accurate diagnosis and/or to prevent first episodes once a person is identified as at risk.

Some processes for diagnosis have been discussed in my patent applications referenced above. One wishes to have activity (motion, verbal responses, positions, etc.) generated by the person being diagnosed which would bring out what you are trying to determine as inappropriate behavior, inordinate motions, or responses to questions or visual social activity by others.

It is generally of interest to divide persons into groups that are being screened. One would be the general population but another group would be those that you suspect may have a potential mental illness based, for example, on input from their parents. Indeed, it may be possible to even have input that would tell what particular type of behavior to be looking for or the first age of screening could be simply to find that out.

The screening clearly should start as early as practicable. One problem with diagnosing illness in young children is that they vary widely in behavior and you really don't know what normal comprehends, particularly as a parent. The video and computer based system is, however, able to process a great deal of data with respect to information obtained from a large number of children and may potentially spot variations that could be significant—a big advantage over “flying blind,” which is the case today. Data can be downloaded to the system for example over the internet, provided on disc or the like.

It is noted that psychiatrists in general don't have anywhere near enough time available to really study their patients. This is even truer with children—especially as it is unlikely the children have been sent to see a psychiatrist. The disclosed invention allows one to accumulate a great deal of data at an early age and if desired, store it in video memory. One can alternatively or in addition, process it after the fact or remotely to look for abnormal behavior. Indeed even a year or two later, if suspicions arise for example, one can, if desired, simply process those sections of data that are viewed as potentially problematic. As one goes forward with this large database of video and related data (again motion, facial expression, voice, position, etc.) one can also input data from parents and caregivers. It would not be out of line for example to have a yearly review of the person, especially if there was any suspicion at all, especially in the case where there is a family history.

If added information is known either from the constant monitoring process or some other data input to the system, one can ask more intelligent questions in the course of interaction with the system. This can be both of the caregiver or parent and of the person via the program. If a person or the program sees something of interest, the program of the system may inquire about it in some form, either directly or potentially indirectly through a game, or other activity.

It should be noted that the well-being of the patient can be improved by also improving their self-esteem and desire to participate in social situations and groups. This is true with weight loss, which is a major issue with those taking certain drugs known to put on weight.

Another place to look for difficulties it is in persons internet chat, particularly video chat, where odd expressions or inappropriate behavior may manifest themselves. This may be because of a feeling by the person that they are not being watched or that they are totally in an informal situation.

Note that psychiatrists generally are pressed for time, and don't have chance to really observe a person. The invention allows gathering of lots of data, including stored video clips and stills and caregiver data. Thus using the vastly increased amount of data, the medical person can ask more intelligent questions, both of the caregiver and the patient allowing better decisions to be made.

Psychosis often affects a person's thinking for example. Everyday thoughts may become confused, they may have difficulty concentrating, following a conversation, or remembering things. Thoughts may seem to speed up or slow down. Using the invention, questions asked or responses naturally requested in an activity such as a video game or internet classroom for example can be monitored to see if anything outside the norm is occurring. Typically motion, pose, voice and expressions (e.g., facial, smile, gesture, etc.) are the main sensed variables.

Some of the many examples are:

Body language, head down, not looking, looking to side, unusual twitches.

Facial expression not matching response called for, such as a frown instead of smile.

Wondering what is going on is another example, manifested as a quizzical look or pose detectable by the camera system of the invention.

Hands shaking and other anxiety or motion disorders.

Schizophrenia problems of motor dysfunction and making inferences of interrelationships.

A deficit in mentalizing or monitoring the intentions of others, which may show up in a story-based comprehension task requiring attribution of mental states to others.

Characteristics of a mood disorder: talking fast or hyperactivity, or conversely lethargic, depressive, uninterested.

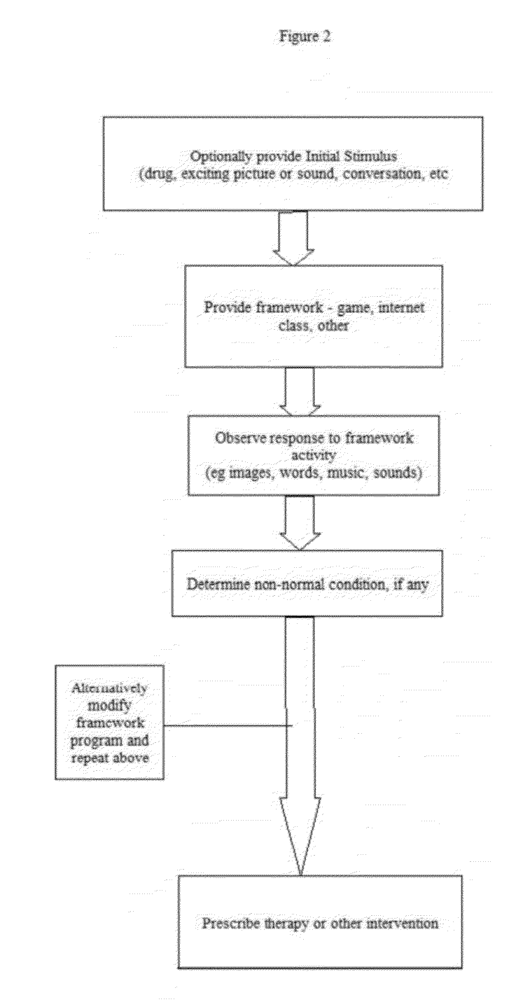

FIG. 2 illustrates one example of one process using the invention to gather data which is analyzed to diagnose a current condition and/or to predict a future condition. One can then repeat the activity every few weeks or as often as it turns out to be necessary and continue to monitor the person throughout a timeframe that could stretch 10 or 15 years for those who might be at risk to develop schizophrenia.

A major benefit of the invention will be realized in those cases where it is possible that lower doses of drugs will be satisfactory, used in conjunction with the invention to provide continual monitoring. This in turn allows users to be more comfortable taking their medication (and living with the side effects, which can be weight gain, shaking, etc.).

The invention may be used to knowledgably identify people at risk and begin giving trace amounts of a new or existing drug, such as an anti-psychotic drug, to forestall problems from occurring.

In some cases the drug may be mixed with small amounts of other drugs to offset negative effects—what and how much is given depending on the degree determined in the game or other activity engaged in using the invention.

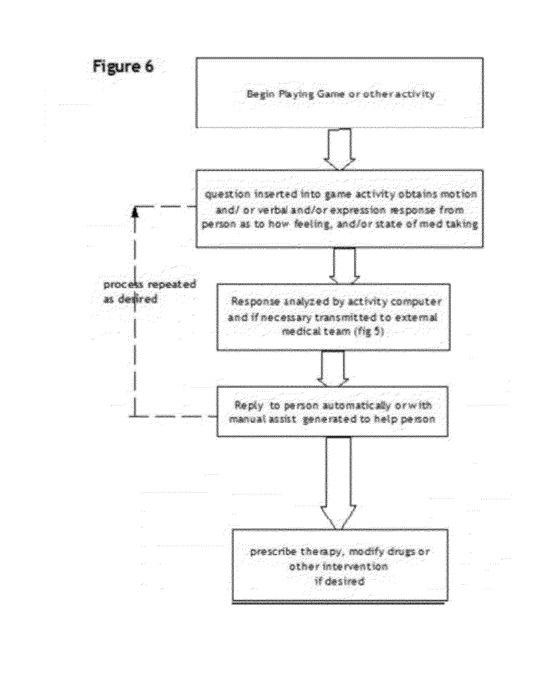

The above comments to this point have been concerned primarily with how to monitor and best optimize medicine being taken by persons who are mentally ill. Another application of the invention is to aid those persons in their daily lives beyond simply staying on and developing the right medical treatment. One sequence of steps in this regard is illustrated in FIG. 6.

A significant problem facing mentally ill people is social interaction. For example, many have no friends and feel that their lives are terrible in a social sense. “Terrible” is the worst level on the standardized questionnaire called the quality-of-life scale, or QOL.

In order to help these people, social situations may, in one example of many, be depicted on the display of the invention. These may be essentially acted out by actors either live or computer graphically generated on the screen. Proper responses to what the actors are saying or doing can be communicated in one form or another to the mentally ill person and accomplished in the privacy of their own home or room, a very important point for some people.

In another form an internet site can be constructed which represents a computer graphic based virtual world that the person can interact in with other virtual persons. Helpful social hints and monitoring would also be utilized to provide feedback. Unlike the Second Life website, in this case no other real persons (other than medical personnel) would typically be interacting. In other cases a form of group therapy might be used where for example two persons could interact in this manner via avatars, which would be moderated by medical personnel or the program of the computer(s) itself.

I have described social scene generation and monitoring in my referenced applications above. A canned video of a social scene could evoke a non-normal response that might be both a diagnostic signal, and a cue to help the person themselves. For example a character's image on the screen could in response say, “what I am really trying to say is this ______.” Or for gesture/position aspects the person could say to the patient, “You should move back, or move closer, or extend your hand” or some such.

It should be noted that the social skills problems may also be used it is a form of diagnosis that persons are tending toward mental illness.

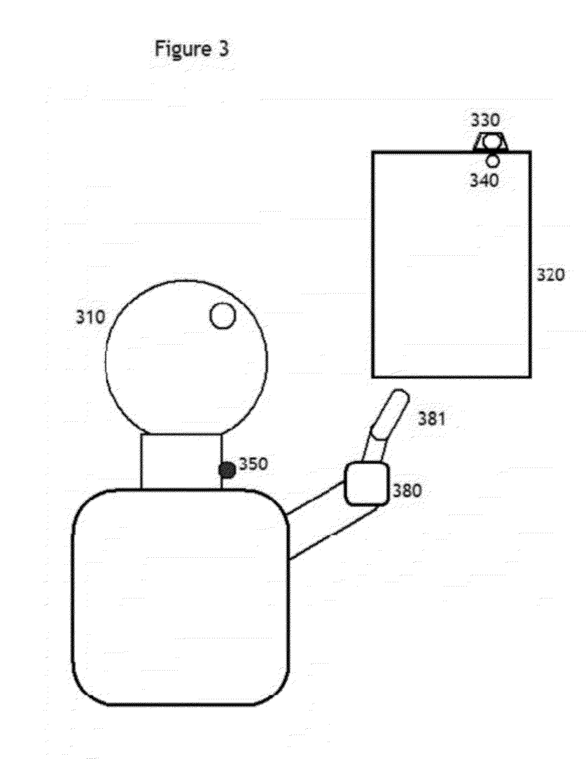

It is also possible to implement the invention in an embodiment which can be portable and even hand-held. For example consider FIG. 3 illustrating use of an iPad 2 tablet by Apple Corporation, 320. In this case, requisite sensing and computational capability for at least determining some movements or gestures, voice, etc., is contained within the portable device which in this case contains both a camera and microphone to pick up video and audio, as well as means to display video and speakers to provide audio information to the person. The computer within the device determines information needed in response the person's actions relating to images or sounds displayed by the device. The camera can be, as in the iPad2, a two dimensional standard type 340, or it can be a special 3D camera. Such a 3D camera may use triangulation via special lighting from an angle (as does today's Microsoft® Kinect™ sensor unit above and described further below), or time of flight principles for example.

In this application the person 310 is typically closer to the screen than in the case shown in FIG. 1 and thus actions visible by the camera are primarily movements and gestures of the head and the upper body. Facial expressions and voice are also able to be monitored, but not the total body movements. This isn't to say however that one couldn't park the iPad on a stand far enough away to see all of these points, but typically one might just simply hold it in one's hand in which case it's primarily the head and upper body that are monitored. Or even smaller features like fingers 381 or hands 380. If one holds the tablet at arm's length or less, one can see the image well and one's voice can be reliably detected from the normal microphone of the device and if desired processed by a voice recognition program such as Apple's SIRI. Generally there is not much problem with background light because the person is occupying most of the camera field making the sensing problem easier. Simple 2D image processing, such as algorithms contained in Matrox MIL or other machine vision packages, can be used to determine locations of features of the person such as tips of fingers, joints in fingers, elbows, heads, shoulders, etc. This sensing can generally be carried out using room lights, but auxiliary lighting may be desired, and suitable light sources such as LEDs provided. High contrast targets such as retro reflector or other targets 350 on the person's neck in this case and illuminated by optional light source 330 attached to the iPad or incorporated into same may be used to make sensing of feature positions and movements easier.

The iPad or other portable device can communicate to external data sources via Wi-Fi, cellular or other means known in the art.

It is noted that there is a close relation between the brain, the hands and the fingers and an embodiment of the invention can be conveniently used to study motion or position response of fingers to inputs such as visual or audio that are processed by the brain. One type of area to determine is willed actions, where correct action such as a finger movement is not evident from context of a spoken command.

While it is not necessary to use a tablet to gain position and/or motion information from the hands and fingers, it is very convenient. A person 730 using a tablet 710 with a 3D camera sensor 720 to see motions, such as motion of hand 740 or one or more fingers thereon, for either mental health or physical rehabilitation purposes is shown in FIG. 3. The person can point or gesture at the screen, which may have the image of a medical person for example displayed, or at some other object in the room or place of interest.

One of the types of images that can be displayed by the invention is that of a caseworker or caregiver who could either be a real person familiar to the patient or of a video of someone standing in for a real person but is actually a live photographed video of a caregiver asking questions. For example in another form could be a computer graphic generated character which gives the added possibility of more types of interactive, dynamic interaction in response to questions or to generate social club movements that might be in response to an inappropriate movement by the person himself. In the case of this close-up iPad application, such inappropriate actions could for example be a facial expression.

The portable device such as a tablet is useful in that the person can carry it with them to their room, and thus be private in its use. It is also preferable in many cases to use your own personal device which has your information, and is not necessary to share in any way with others. The Tablet has all necessary functions for display, transmission, audio and computation and can offer a complete solution on an individualized basis. Cell phones can too, but display is limited in size.

The portable devices of FIGS. 3 and 4 have an advantage wherein they can allow an ease of interaction by the user, which may often advantageously be done in private. There can be games loaded in both phones and tablets that have some motion complement if desired. One can use these devices as illustrated in FIG. 1 for internet classes or web chats as well. It doesn't have to be a game per se that is participated in during a monitoring session.

FIG. 4 illustrates a further portable application of the invention, which could be in an iPad tablet type of arrangement is in FIG. 3, but is shown here in even more handheld fashion as part of an iPhone, or other smartphone 440. Again, points relating to movement of the person 410 looking at display 400 may be electro-optically monitored by sensor 430 in conjunction with computer 420 which also controls the TV display 400, but in this case the movement may be of the phone 440 in the hands of the person 410 (as illustrated in my U.S. Pat. No. 7,933,431, incorporated herein by reference) rather than the person relative to the phone. Alternatively for example, the movement of a phone 440 may be sensed by an accelerometer within it as jiggled, shaken or jerked and this information communicated to the computer 420, and if desired to external locations. In one example the person may be shaking due to Parkinsonian symptoms. Data 450 may optionally be transferred to and from the phone 440 to the computer 420.

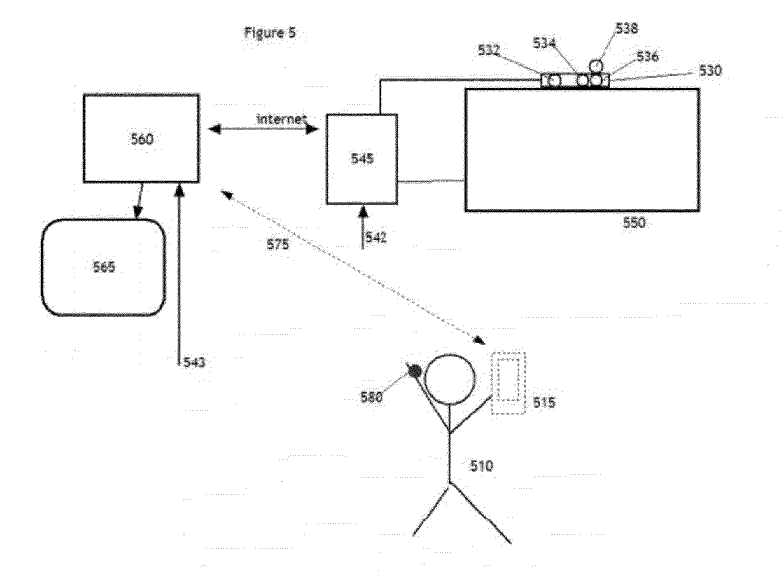

In operation the person participates in an activity, which can be a simple iPad or iPhone game. However the motion input, video and voice of the person is analyzed by the computer of the device and/or transmitted to a remote location for analysis in the embodiments above sensed or other input data can be transmitted to a remote location or sensed or taken from the patient and communicated to the patient. This is further illustrated in FIG. 5.

FIG. 3 and FIG. 5 have both illustrated optional light sources which can be used to illuminate the region of the person participating in the activity. The reason for this is to allow better definition of points, which can be retro-reflective as shown in the drawings, if the light source is placed near the camera. In the case of FIG. 3, the light source can be white light or other color such as a deep red light which can still pass the IR blocking filter of the conventional Tablet. A 660 nm LED will work in this regard, though a deeper red is still better, if available. Even better is to use an IRLED, but this involves the fitting of a different IR cut filter to the camera than is normally provided. If deep red or IR it is less visible and thus less objectionable to the user looking at it, though in some cases it may be desired that the user see the points being specifically illuminated. It should also be noted that the efficiency of even glass bead retro-reflectors (e.g., Scotchlight brand by 3M corporation) that the light source may be relatively dim while still allowing the retro-reflectors to be accurately seen by the camera at speeds of 30 frames per second or more. Unless the retro-reflector indications are processed out of the displayed image (as disclosed in co-pending applications of mine) the target points will show, but will not in this case appear super bright (and sometimes blooming) and perhaps objectionable in that regard. It is noted too that the target images can be sensed by the machine vision program in the tablet (cell phone, etc.) and colored in for example by the display program. For example they could be green if moved to the correct location (which could be taught location) and colored red if they were falling short.

In the drawing of FIG. 5, a Microsoft® Kinect™ sensor 530 is controlled by a computer 545 which controls the display 550 and optionally connects via the internet or other means to a remote computer 560 having a display 565. The Kinect™ sensor in this example obtains movement position and audio data concerning a person 510 who may optionally be holding an object such as cell phone 515. This cell phone may be connected via Wi-Fi or Bluetooth for example to the computer 545, or may be connected (575, dotted lines) over the cellphone or other network to remote to a computer 560. The camera section of the Microsoft® Kinect™ utilizes a projector 532 of a random IR grid of points to triangulate range data using a camera 536 having a narrow band pass filter in front of the lens (not shown for clarity) to pass the IR wavelength of the laser, illuminating the laser illuminated diffractive element, and creating the grid. The Kinect™ sensor further employs a webcam 534 to obtain color imagery concerning the person 510.

In FIG. 5, I have included in the sensing apparatus an additional solid state light source such as an IR LED or IR laser 538. The solid state source has substantially the same wavelength as the laser used for range data gathering and is used to illuminate retro-reflective or other discrete points on a person or elsewhere, such as retro reflector 580. This allows the camera 536 to get reliable definition of such points, in fast motion. Contrast is high when the source 538 is near the optical axis of the camera used to obtain data from retroreflective targets. This solid state source can be turned off or attenuated as necessary when the camera is used to view the grid image triangulated (or other range image such as that generated by a time of flight based camera such as that of Canesta corporation). The bandpass filter pass band on camera 536 may have to be broader when an additional LED is used, rather than a laser.

As an alternate to using the range image camera, the associated RGB camera (webcam) 534 can be used to sense discrete points in a manner similar to the technique of FIG. 3. An auxiliary LED at a wavelength suitable to be sensed by the webcam may be used. This wavelength may need to be in the visible range if an IR cut filter is employed by the webcam.

Use of defined points allows precise data as to movements of body portions such as finger movements for example to be obtained, and may optionally be incorporated.

Processing for diagnosis or other purposes can be from afar if image video and voice transmitted. Generally one only need to be real time connected (local or high speed connection) if instant reaction of the computer or medical personnel to the activity of the person monitored is needed.

More than one person can interact with a remote or computer stored caseworker type person as well. In a sense this is a form of group therapy over the internet. While in its basic form it doesn't need motion sensing to get started, but this and other automatically sensed functions (verbal, facial, etc.) can be incorporated. I give an example in a related area in my above referenced '404 application having a live exercise bicycle spinning instructor with multiple people in the group. In this case the instructor can't watch all in the group at once, but the camera in each individual's system can obtain images and information for the instructor to view when possible or called for as it can in this case, with a goal of helpful therapy for individuals either during the session or after, depending on what is discovered.

Other data such as medical data 542 may be provided to the computer 545 to aid in obtaining information concerning the person. Similarly other such data 543 can be provided to the remote computer 560, which might be located at a medical practitioner's or caseworker's office.

Reinforcement of ideas may be suggested during the computer activity, such as a game. One can “nudge” the person into adapting socially acceptable or other proper behavior. This can be done, for example, relative to taking medicine by playing a game where such is rewarded in the outcomes for others in the virtual world, or real rewards of virtual items are earned if one confirms they took their medications, took them on time, in the correct dosage, etc.

It is noted that teaching of various dance steps, movements, social responses, and the like can be done with the aid of a social worker or other medical person and the positions movements or even voice recorded of the person, when so aided. Then at a future time, the person can replay the taught sequences and check their progress using graphical or other aids if desired, with the comfort of seeing the teacher on the screen with them.

### Mental Health for Seniors, Stroke Victims and Others

I feel the invention can also help senior citizens to provide an early diagnosis of Alzheimer's or other dementia-related illnesses. This comes from the response to questions that may be posed in the course of a normal activity. Senior games which incorporate motion can focus more on the upper body area (and be played sitting down if desired) and can be more focused on hand dexterity as a precursor to arthritis. Sometimes people don't report things as soon as they should, and if they don't mind being given a suggested diagnoses, this can be valuable. It also can form a record of their health.

Cognitive relearning treatment programs can be aided by the invention which can help them start as soon as possible by making the activity fun and more motivational.

A real person or a video clip of a real person looking at you via the TV and asking a question I believe is still better than a computer graphic, though these get more lifelike every day. Perhaps for some people the fact they don't look totally real might be a plus.

Alternatively the diagnosis from the images and sounds recorded can be done after the fact unless response of the person on the screen needs to be completely interactive to provide help and well-being to the user. And I believe that only if you do it in real time, can you get really good question and response.

Various places other than the home may be used to gather data, also where one might have in the course thereof the person sign a waiver with respect to use of the data. For example playing games in a pediatrician's office, school activity, armed forces, camp physicals, online activity with licensed software, etc.

### Aid to Community Support

The invention provides motivation for the user and data for health care professionals, further providing a means of communication to them as well as to caregivers, all at the lowest possible cost to enable mass usage by patients as well as therapists, doctors and clinicians. Let's look at an example. A person with mental illness may use the device in their home to keep in daily contact with health care providers. Once a day they may chat and discuss things, and then at other random times through the day data is obtained automatically whenever the person uses the device for gaming, chatting or other purposes. This data may be all accumulated to aid the caregiver on the scene (if any) and the remote care providers. By use of both the manual remote version and the automatic data gathering a much higher level of care can be provided, assuming the person uses the device. For this reason, incentive for use is likely needed. One incentive is social interaction made possible, such as virtual or real interaction sites on the internet (which might be run by the community). Another could be monetary rewards. Fun activities such as video games are another.

In the course of activity, the computer program of the invention may determine that the person playing the activity is in need of help. The computer can call or otherwise signal caregivers using known means. It can also provide therapeutic signals suggestions and the like for the person directly. For example, this can be calming music and/or images if excessive anxiety or manic activity is determined. It could be asking questions which would further help identify the right personnel to intervene. One can show images and recorded voices as well of a favorite caregiver or caseworker as examples. It could automatically initiate stress relieving exercise, such as for some, Tai Chi.

While the invention has been described in connection with numerous embodiments, it is to be understood that the specific mechanisms and techniques that have been described are merely illustrative of the principles of the invention, and numerous modifications may be made to the methods and apparatus described without departing from the spirit and scope of the invention.

1. A method for providing improved treatment of mental illness comprising the steps of:
providing a computer based system to sense information concerning a person's activity and to provide visual and audio information to said person;
using said system, obtaining data concerning said patient during the activity undertaken;
analyzing said data; and
from said analysis, diagnosing the person or determining effectiveness of a current mental health related treatment plan.

2. A method according to claim 1 wherein said activity is a normal activity of the person.

3. A method according to claim 1 further including the step of providing, based on said diagnosis, drugs or other interventions to prevent the occurrence of a first psychotic episode, or to mitigate the effects thereof.

4. A method according to claim 1 wherein audio or video stimuli are further provided to illicit possible responses characteristic of a mental illness.

5. A method according to claim 1 wherein at least a portion of said information is obtained electro-optically.

6. A method according to claim 5 wherein data obtained concerns patient movements.

7. A method according to claim 5 wherein data obtained concerns gestural or facial expressions.

8. A method according to claim 1 wherein said activity includes a social activity.

9. A method according to claim 1 further including the step of recording said data for future analysis.

10. A method according to claim 1 further including the steps of transmitting said data for a remote location for analysis and visual or audio information back to the person while they are engaged in said activity.

11. Apparatus for obtaining information concerning a person or object comprising:
a sensing system including a camera adapted to sense at least one range point on said person or object, said camera having an output and responsive to light substantially at a first wavelength, and said system further including a first solid state light source to illuminate said points at said first wavelength;
a second solid state light source to illuminate one or more points on said person or object independently of said first solid state light source; and
a computer for determining, using said camera output, information concerning said person or object.

12. Apparatus according to claim 11 wherein said camera is further adapted to sense points other than range points on said person or object.

13. Apparatus according to claim 12 wherein at least one of the points illuminated by said second light source is retro-reflective and said second light source is located in close proximity to said camera.

14. Apparatus according to claim 11 wherein said second light source is substantially operating at a wavelength similar to said first wavelength.

15. Apparatus according to claim 11 wherein said sensing system and said second light source are contained within a housing.

16. Apparatus according to claim 11 wherein said first and second light sources are illuminated at different times.

17. Apparatus according to claim 11 wherein said first and second light sources are diode lasers.

18. Apparatus according to claim 11 wherein said second light source is an LED.

19. Apparatus according to claim 11 further including a second camera having an output, said computer determining information using the outputs of said first and second cameras and said second camera being responsive to light from at least said second solid state light source.

20. A method for obtaining information concerning a person or object, comprising the steps of:
providing a camera system for obtaining data concerning range points on said person or object;
providing a further camera;
providing a solid state light source located proximate said further camera;
providing a computer to process data obtained from said range camera and said further camera;
illuminating at least one retro-reflective target on said person or object; and
using said computer, processing data obtained and determining therefrom information from at least one retro-reflective target and at least one range point.
